(19) United States
(12) Patent Application Publication
(10) Pub. No.: US 2013/0178680 A1
(43) Pub. Date: Jul. 11, 2013

(54) CATALYST FOR OXIDATIVE COUPLING OF METHANE, METHOD FOR PREPARING THE SAME, AND METHOD FOR OXIDATIVE COUPLING REACTION OF METHANE USING THE SAME

(71) Applicant: Korea Institute of Science and Technology, Seoul (KR)

(72) Inventors: Jeong Myeong HA, Seoul (KR); Dong Jin SUH, Seoul (KR); Jae Wook CHOI, Incheon (KR); Young Hyun YOON, Seoul (KR); Gi Seok YANG, Seoul (KR); Wonjin JEON, Seoul (KR)

(73) Assignee: KOREA INSTITUTE OF SCIENCE AND TECHNOLOGY, Seoul (KR)

(21) Appl. No.: 13/668,516

(22) Filed: Nov. 5, 2012

(30) Foreign Application Priority Data
Jan. 11, 2012 (KR) 10-2012-0003391

(51) Int. Cl. B01J 23/34; C07C 2/84

(52) U.S. Cl. 585/541; 585/658; 585/700; 502/306

(57) ABSTRACT

The present disclosure relates to a catalyst for oxidative coupling of methane, specifically, it relates to a catalyst for oxidative coupling of methane comprising: a magnesium titanium oxide support comprising a mixed oxide of magnesium and titanium; and sodium tungstate and manganese oxide supported on the support, a method for preparing the same, and a method for oxidative coupling of methane. The catalyst for oxidative coupling according to the present disclosure, wherein a mixed oxide of magnesium and titanium is used as the support of the catalyst, is capable of providing significantly improved catalytic activity and C₂ hydrocarbon yield as compared to pure magnesium oxide or titanium oxide. By preparing the oxide support not by a physical process but by a chemical sol-gel process, a catalyst for oxidative coupling with a peculiar crystal structure not found in a single oxide support can be provided.

### CROSS-REFERENCE TO RELATED APPLICATIONS

This application claims priority under 35 U.S.C. §119 to Korean Patent Application No. 10-2012-0003391 filed on Jan. 11, 2012, in the Korean Intellectual Property Office, the disclosure of which is incorporated herein by reference in its entirety.

### TECHNICAL FIELD

The present disclosure relates to a catalyst for oxidative coupling of methane capable of achieving high C₂ hydrocarbon yield in oxidative coupling of methane, a method for preparing the same, and a method for oxidative coupling of methane using the same.

### BACKGROUND

Methane, which is the main component of natural gas, is a useful natural resource that can be used as fuel or converted into valuable products through chemical processes. For example, methane can be used as fuel in power plants, industrial facilities and households or can be converted into various compounds such as olefin through direct or indirect conversion. Recently, production of petrochemical products by converting methane is gaining importance.

There are two methods of converting methane. The first is a method of converting methane into a synthesis gas (H₂ and CO) by a methane reforming process such as steam reforming, oxy-reforming, carbon dioxide reforming (CO₂-reforming), etc. and obtaining petrochemical products through subsequent conversion. This method, called indirect conversion, is disadvantageous in that a lot of energy is required to obtain the synthesis gas and initial investment cost is high. The second is a method of directly converting methane into desired hydrocarbon materials without passing intermediates such as the synthesis gas. This method is called direct conversion. Although it is advantageous in that the final product can be obtained through a single process, the method is economically less feasible than the indirect conversion method for now. To overcome this limitation, new catalysts are being actively developed.

The representative direct conversion method of methane is oxidative coupling of methane. This method is useful in that C₂ hydrocarbons can be obtained from methane, which is the main component of natural gas. Scheme 1 describes conversion of methane to a C₂ hydrocarbon ethylene via oxidative coupling:

2CH₄+O₂→C₂H₄+2H₂O <Scheme 1>

As seen from above, oxidative coupling of methane is advantageous in that C₂ hydrocarbons can be obtained from a relatively simple reaction system without additional treatment processes. However, since this method is economically less feasible than the indirect conversion method for now (For example, in the conversion to ethylene described above, methane tends to be oxidized to carbon dioxide rather than to be converted to ethylene.), as described earlier, development of a catalyst capable of increasing selectivity for C₂ hydrocarbons is necessary. In addition, since the existing catalysts require high temperatures, development of a catalyst with a low activation temperature is also important.

At present, a catalyst in which sodium tungstate and manganese oxide are supported on a metal oxide support, for example, is used for oxidative coupling of methane. For example, Korean Patent Publication No. 2010-130722 discloses a silica aerogel-supported catalyst and a method for converting methane using the same. Specifically, it discloses a silica aerogel support and a silica aerogel-supported catalyst in which sodium tungstate and manganese oxide are supported on the support.

### SUMMARY

The present disclosure is directed to providing a catalyst for oxidative coupling of methane providing high C₂ hydrocarbon yield and selectivity, a method for preparing the same, and method for oxidative coupling of methane using the same.

In an aspect, the present disclosure provides a catalyst for oxidative coupling of methane comprising: a magnesium titanium mixed oxide support comprising mixed oxide of magnesium and titanium; and sodium tungstate and manganese oxide supported on the support.

In an exemplary embodiment of the present disclosure, the magnesium titanium mixed oxide support may comprise magnesium and titanium at a molar ratio of from 1:9 to 9:1.

In another exemplary embodiment of the present disclosure, the magnesium titanium mixed oxide support may comprise magnesium and titanium at a molar ratio of 5:5.

In another exemplary embodiment of the present disclosure, the sodium tungstate may be included in an amount of 1-10 wt % and the manganese oxide may be included in an amount of 1-5 wt % based on the total weight of the catalyst.

In another exemplary embodiment of the present disclosure, the sodium tungstate may be included in an amount of 3-5 wt % and the manganese oxide may be included in an amount of 1-3 wt % based on the total weight of the catalyst.

In another aspect, the present disclosure provides a method for preparing a catalyst for oxidative coupling of methane, comprising:

preparing a solution in which magnesium alkoxide and titanium alkoxide are dissolved in an organic solvent;

inducing hydrolysis and condensation by adding deionized water while stirring the solution, adding a sodium tungstate precursor and a manganese oxide precursor and further stirring until the solution transforms into a gel; and

drying and baking the gel.

In an exemplary embodiment of the present disclosure, the hydrolysis and the condensation may be performed at pH 2-5.

In another exemplary embodiment of the present disclosure, the hydrolysis, the condensation and the gelation may be performed at normal temperature and normal pressure for 6-24 hours.

In another exemplary embodiment of the present disclosure, the drying may be performed at a pressure of 1 torr or lower and the baking may be performed at 500-900° C.

In another aspect, the present disclosure provides a method for oxidative coupling of methane, comprising:

packing the catalyst for oxidative coupling of methane in a reactor; and

performing oxidative coupling of methane by introducing a gas mixture comprising methane, oxygen and an inert gas into the reactor.

In an exemplary embodiment of the present disclosure, the temperature inside the reactor may be maintained at 600-900° C.

In another exemplary embodiment of the present disclosure, the temperature inside the reactor may be maintained at 700-850° C.

In another exemplary embodiment of the present disclosure, the volume ratio of oxygen and methane in the gas mixture may be from 1:1 to 1:10.

In another exemplary embodiment of the present disclosure, the volume ratio of oxygen and methane in the gas mixture may be from 1:2 to 1:5.

In another exemplary embodiment of the present disclosure, the inert gas may be helium gas and may be included in the gas mixture in an amount of 10 -30 vol %.

In another exemplary embodiment of the present disclosure, the gas hourly space velocity (GHSV) of the gas mixture inside the reactor may be 5,000-30,000 h⁻¹.

The catalyst for oxidative coupling according to the present disclosure, wherein a mixed oxide of magnesium and titanium is used as the support of the catalyst, is capable of providing significantly improved catalytic activity and C₂ hydrocarbon yield as compared to when pure magnesium oxide or titanium oxide is used as the support. By preparing the oxide support not by a physical process but by a chemical sol-gel process, a catalyst for oxidative coupling with a peculiar crystal structure not found in a single oxide support can be provided.

### DETAILED DESCRIPTION OF EMBODIMENTS

A catalyst for oxidative coupling of methane according to the present disclosure comprises: a magnesium titanium oxide support comprising a mixed oxide of magnesium and titanium; and sodium tungstate and manganese oxide supported on the support. That is to say, the catalyst according to the present disclosure uses a magnesium titanium oxide support comprising a mixed oxide of magnesium and titanium as a support and sodium tungstate and manganese oxide catalyzing oxidative coupling are supported on the support.

As demonstrated in the examples to be described later, the magnesium titanium oxide support may lead to better catalytic activity and C₂ hydrocarbon yield than when pure magnesium oxide or titanium oxide is used. As used in the present disclosure, ‘C₂ hydrocarbon’ collectively refers to hydrocarbons having two carbon atoms including, but not being limited to, ethane, ethylene or acetylene.

In the support, the molar ratio of magnesium to titanium may be from 1:9 to 9:1, more specifically 5:5. In the present disclosure, a support comprising a single oxide is structurally modified so as to reorganize the lattice structure of the support and thus to induce structural imbalance of electronic distribution. Specifically, since magnesium ion is a divalent cation and titanium ion is a monovalent cation, structural imbalance of electronic distribution may be induced when the magnesium ion in magnesium oxide is replaced with a titanium ion. Also, since the two atoms have different physical properties, structural defect occurs during synthesis and baking when magnesium oxide and titanium oxide exist together. The structural imbalance of electronic distribution and structural defect are important factors determining the acidity or basicity of the catalyst. Especially, the basicity of magnesium titanium oxide changes with the mixing ratio of magnesium and titanium. It is reported that the reaction activity and O₂ hydrocarbon yield in oxidative coupling of methane increase as the basicity of the catalyst increases. The inventors of the present disclosure have experimentally compared C₂ hydrocarbon yield in oxidative coupling of methane for different mixing ratios of magnesium and titanium. As a result, they have found out that C₂ hydrocarbons are produced at the highest yield when the two metal atoms are mixed at a molar ratio of from 1:9 to 9:1, more specifically 5:5.

In the catalyst according to the present disclosure, sodium tungstate and manganese oxide are supported on the support as catalyst components. The supporting amount of the catalyst components may be 1-10 wt % and 1-5 wt %, more specifically 3-5 wt % and 1-3 wt %, respectively, based on the total weight of the catalyst. When the supporting amounts of the sodium tungstate and the manganese oxide are smaller, they may not effectively function as catalysts in oxidative coupling of methane. And, even when the supporting amounts exceed the above-described ranges, a better catalytic activity is not achieved as compared to when they are supported in amounts within the above-described ranges.

A method for preparing a catalyst for oxidative coupling of methane according to the present disclosure comprises: preparing a solution in which magnesium alkoxide and titanium alkoxide are dissolved in an organic solvent; inducing hydrolysis and condensation by adding distilled water while stirring the solution, adding a sodium tungstate precursor and a manganese oxide precursor and further stirring until the solution transforms into a gel; and drying and baking the gel.

After magnesium alkoxide and titanium alkoxide are hydrolyzed and condensed, a magnesium titanium oxide support is obtained. The magnesium oxide and the titanium oxide may be included at a molar ratio of from 1:9 to 9:1. The sodium tungstate precursor and the manganese oxide precursor are added in amounts such that the final supporting amounts of sodium tungstate and manganese oxide after the drying and baking are 1-10 wt % and 1-5 wt %, more specifically 3-5 wt % and 1-3 wt %, respectively, based on the total weight of the catalyst.

The hydrolysis and the condensation conducted by adding deionized water to the solution may be performed at pH 2-5. At pH<2, it takes a very long time until sol particles are formed or, even if the sol is formed, it may not be transformed into gel but precipitate. And, at pH>5, gel particles may form partially in the state where hydrolysis has not occurred sufficiently.

The hydrolysis, the condensation and the gelation are performed at normal temperature and normal pressure for 6-24 hours since the rate of hydrolysis and condensation depends on the solution pH. After the gelation is completed, the gel is dried and baked to obtain the catalyst for oxidative coupling according to the present disclosure. The drying may be performed at a pressure of 1 torr or lower to obtain a xerogel which is a porous dry gel, and the baking may be performed at 500-900° C. in order to obtain a surface crystal structure having reaction activity.

The present disclosure further provides a method for oxidative coupling of methane using the catalyst for oxidative coupling prepared above. Specifically, the method for oxidative coupling of methane according to the present disclosure comprises: packing the catalyst for oxidative coupling of methane according to the present disclosure in a reactor; and performing oxidative coupling of methane by introducing a gas mixture comprising methane, oxygen and an inert gas into the reactor.

The catalyst for oxidative coupling of methane according to the present disclosure may be packed along with an inert packing material in, for example, a quartz reactor having appropriate inner diameter and length. The catalyst may be pretreated at high temperature in order to remove moisture and impurities included in the catalyst. The treatment may be performed, for example, at 600-900° C. for about 1 hour in the presence of an inert gas such as helium.

After the treatment is completed, a gas mixture comprising methane, oxygen and an inert gas is introduced into the reactor. As described in the following examples, the inventors of the present disclosure have tested the performance of the catalyst for various volume ratios of oxygen to methane in the reactant. As a result, they have found out that the highest catalytic activity and C₂ hydrocarbon yield are achieved when the volume ratio of oxygen and methane in the gas mixture is from 1:1 to 1:10, more specifically from 1:2 to 1:5. The remaining volume of the reactant excluding those of methane and oxygen is filled with the inert gas such as helium gas in order to ensure reaction stability. Specifically, the inert gas may be included in the gas mixture in an amount of 10 -30 vol %.

In the present disclosure, the gas hourly space velocity (GHSV) of the gas mixture inside the reactor may be 5,000-30,000 h⁻¹. The gas hourly space velocity is the entering volumetric flow rate of the reactant divided by the catalyst bed volume. The larger the gas hourly space velocity, the shorter the reactant remains in the catalyst bed. The packing amount of the catalyst according to the present disclosure in the reactor may be different depending on the catalyst since different catalyst has different density, and may be calculated based on the gas hourly space velocity of the reactant, i.e. the gas mixture, and the flow rate of the reactant.

The temperature inside the reactor may be maintained at 600-900° C., more specifically at 700-850° C. When compared with the existing art, the present disclosure is advantageous in that energy cost can be saved since the catalyst performance can be exhibited at relatively lower temperatures.

### EXAMPLES

Hereinafter, the present disclosure will be described in further detail through examples. However, the following examples are for illustrative purposes only and should not be interpreted to limit the scope of this disclosure.

### Example 1

### Preparation of Catalyst According to the Present Invention

Alkoxides of each oxide Ti(OC₂H₅)₄ and Mg(OC₂H₅)₂ were dissolved respectively in an organic solvent (ethanol) and mixed such that the molar ratio of magnesium to titanium was 25:75, 50:50 or 75:25 (mol %). Hydrolysis and condensation were performed by adding distilled water while constantly stirring the mixture solution. During this procedure, the catalyst precursors Na₂WO₄.2H₂O and Mn(NO₃)₂.6H₂O were added in an amount of 5 wt % and 2 wt %, respectively, based on the total weight of the catalyst. The solution was stirred until it transformed into a gel while maintaining the pH of the solution at 3 using nitric acid and aqueous ammonia. According to a previous report, at lower pH, the gelation rate decreases but the surface area of the mixed oxide product increases. The produced gel was dried at 105° C. for 24 hours and baked at 800° C. for about 5 hours to obtain a catalyst. The baking was performed under air atmosphere.

### Comparative Example 1

A catalyst comprising magnesium oxide and titanium oxide at a ratio of 0:100 or 100:0 was prepared in the same manner as in Example 1 using only Ti(OC₂H₅)₄ or Mg(OC₂H₅)₂.

### Comparative Example 2

A catalyst of the same composition was prepared in the same manner as in Example 1 using a silica (SiO₂) support.

### Test Example 1

The catalyst according to the present disclosure prepared in Example 1 was fixed inside a quartz reactor. After pretreating at 700° C. with nitrogen, a gas mixture of methane and oxygen was supplied at a ratio of 5:1. While increasing temperature from 700° C. to 850° C. with 25° C. intervals, the products of each temperature zone was analyzed after reaction for 40 minutes. The result is shown in Table 1. Table 1 shows a result when the mixing ratio of magnesium oxide and titanium oxide were 25:75, 50:50 and 75:25 (mol %).

TABLE 1

| | | | |
|---|---|---|---|
| Reaction | Conversion ratio of | C₂ hydrocarbon | C₂ hydrocarbon |
| temperature (° C.) | methane (%) | selectivity (%) | yield (%) |

Mg:Ti = 25:75 (mol %)

| | | | |
|---|---|---|---|
| 725 | 2.8 | 23.5 | 0.6 |
| 750 | 9.9 | 23.9 | 2.3 |
| 775 | 22.6 | 37.5 | 8.5 |
| 800 | 34.0 | 40.2 | 13.7 |
| 825 | 37.5 | 40.6 | 15.2 |
| 850 | 39.1 | 41.3 | 16.1 |

Mg:Ti = 50:50 (mol %)

| | | | |
|---|---|---|---|
| 725 | 11.2 | 27.5 | 3.1 |
| 750 | 25.7 | 41.9 | 10.8 |
| 775 | 37.1 | 44.6 | 16.5 |
| 800 | 41.4 | 43.1 | 17.9 |
| 825 | 42.4 | 42.7 | 18.1 |
| 850 | 42.4 | 41.7 | 17.7 |

Mg:Ti = 75:25 (mol %)

| | | | |
|---|---|---|---|
| 725 | 1.4 | 31.1 | 0.4 |
| 750 | 4.3 | 37.2 | 1.6 |
| 775 | 9.2 | 50.6 | 4.6 |
| 800 | 15.8 | 61.8 | 9.8 |
| 825 | 40.9 | 44.0 | 18.0 |
| 850 | 42.0 | 41.0 | 17.2 |

### Comparative Test Example 1-1

Experiment was carried out under the same condition as in Test Example 1 except for using a sodium tungstate-manganese oxide catalyst supported on a support comprising magnesium oxide only. The result is shown in Table 2.

TABLE 2

| Reaction | Conversion ratio of | C₂ hydrocarbon | C₂ hydrocarbon |
|---|---|---|---|
| temperature (° C.) | methane (%) | selectivity (%) | yield (%) |
| 725 | 3.8 | 11.2 | 0.4 |
| 750 | 4.9 | 34.9 | 1.7 |
| 775 | 10.6 | 40.2 | 4.3 |
| 800 | 11.9 | 53.1 | 6.3 |
| 825 | 16.3 | 56.9 | 9.3 |
| 850 | 29.8 | 50.8 | 15.1 |

### Comparative Test Example 1-2

Experiment was carried out under the same condition as in Test Example 1 except for using a sodium tungstate-manganese oxide catalyst supported on a support comprising titanium oxide only. The result is shown in Table 3.

TABLE 3

| Reaction | Conversion ratio of | C₂ hydrocarbon | C₂ hydrocarbon |
|---|---|---|---|
| temperature (° C.) | methane (%) | selectivity (%) | yield (%) |
| 725 | 1.4 | 28.6 | 0.4 |
| 750 | 2.1 | 47.4 | 1.0 |
| 775 | 5.0 | 49.5 | 2.4 |
| 800 | 11.8 | 51.1 | 6.0 |
| 825 | 23.9 | 51.4 | 12.3 |
| 850 | 37.3 | 45.6 | 17.0 |

### Comparative Test Example 1-3

Experiment was carried out under the same condition as in Test Example 1 except for using a sodium tungstate-manganese oxide catalyst supported on a support comprising silicon oxide only. The result is shown in Table 4.

TABLE 4

| Reaction | Conversion ratio of | C₂ hydrocarbon | C₂ hydrocarbon |
|---|---|---|---|
| temperature (° C.) | methane (%) | selectivity (%) | yield (%) |
| 725 | 1.7 | 35.6 | 0.6 |
| 750 | 10.6 | 32.3 | 3.4 |
| 775 | 26.5 | 48.0 | 12.8 |
| 800 | 41.8 | 43.8 | 18.3 |
| 825 | 43.4 | 42.3 | 18.4 |
| 850 | 43.7 | 39.8 | 17.4 |

### Comparative Test Example 1-4

In order to compare the catalytic activity of the catalyst that exhibited the best activity in Test Example 1 (magnesium:titanium=50:50 mol %) with that of a catalyst supported on silicon oxide, experiment was performed at a gas hourly space velocity (GHSV) of 10,000 h⁻¹. Conditions other than the space velocity were the same as in Test Example 1. The result is shown in Table 5.

TABLE 5

| | | | |
|---|---|---|---|
| Reaction | Conversion ratio of | C₂ hydrocarbon | C₂ hydrocarbon |
| temperature (° C.) | methane (%) | selectivity (%) | yield (%) |

Mg:Ti = 50:50 (mol %)

| | | | |
|---|---|---|---|
| 725 | 19.3 | 27.2 | 5.2 |
| 750 | 29.5 | 35.4 | 10.4 |
| 775 | 35.6 | 39.2 | 14.0 |
| 800 | 38.3 | 40.5 | 15.5 |
| 825 | 39.5 | 40.1 | 15.9 |
| 850 | 39.7 | 38.9 | 15.5 |

Catalyst supported on pure SiO₂

| | | | |
|---|---|---|---|
| 725 | 9.0 | 87.0 | 2.4 |
| 750 | 20.7 | 62.1 | 7.5 |
| 775 | 31.7 | 53.1 | 13.3 |
| 800 | 38.9 | 48.7 | 16.4 |
| 825 | 41.2 | 48.3 | 16.8 |
| 850 | 41.0 | 49.1 | 16.0 |

### Test Example 2

Experiment was carried out under the same condition as in Test Example 1 except for changing the ratio of methane to oxygen to 2:1. The result performing oxidative coupling with different oxide composition is shown in Table 6.

TABLE 6

| | | | |
|---|---|---|---|
| | Conversion ratio of | C₂ hydrocarbon | C₂ hydrocarbon |
| Mg:Ti (mol %) | methane (%) | selectivity (%) | yield (%) |

Reaction temperature = 725° C.

| | | | |
|---|---|---|---|
| 0:100 | 1.4 | 28.6 | 0.4 |
| 25:75 | 2.8 | 23.5 | 0.6 |
| 50:50 | 11.2 | 27.5 | 3.1 |
| 75:25 | 1.4 | 31.1 | 0.4 |
| 100:0 | 3.8 | 11.2 | 0.4 |

Reaction temperature = 750° C.

| | | | |
|---|---|---|---|
| 0:100 | 2.1 | 47.4 | 1.0 |
| 25:75 | 9.9 | 23.9 | 2.3 |
| 50:50 | 25.7 | 41.9 | 10.8 |
| 75:25 | 4.3 | 37.2 | 1.6 |
| 100:0 | 4.9 | 34.9 | 1.7 |

Reaction temperature = 775° C.

| | | | |
|---|---|---|---|
| 0:100 | 5.0 | 49.5 | 2.4 |
| 25:75 | 22.6 | 37.5 | 8.5 |
| 50:50 | 37.1 | 44.6 | 16.5 |
| 75:25 | 9.2 | 50.6 | 4.6 |
| 100:0 | 10.6 | 40.2 | 4.3 |

Reaction temperature = 800° C.

| | | | |
|---|---|---|---|
| 0:100 | 11.8 | 51.1 | 6.0 |
| 25:75 | 34.0 | 40.2 | 13.7 |
| 50:50 | 41.4 | 43.1 | 17.9 |
| 75:25 | 15.8 | 61.8 | 9.8 |
| 100:0 | 11.9 | 53.1 | 6.3 |

Reaction temperature = 825° C.

| | | | |
|---|---|---|---|
| 0:100 | 23.9 | 51.4 | 12.3 |
| 25:75 | 37.5 | 40.6 | 15.2 |
| 50:50 | 42.4 | 42.7 | 18.1 |
| 75:25 | 40.9 | 44.0 | 18.0 |
| 100:0 | 16.3 | 56.9 | 9.3 |

Reaction temperature = 850° C.

| | | | |
|---|---|---|---|
| 0:100 | 37.3 | 45.6 | 17.0 |
| 25:75 | 39.1 | 41.3 | 16.1 |
| 50:50 | 42.4 | 41.7 | 17.7 |
| 75:25 | 42.0 | 41.0 | 17.2 |
| 100:0 | 29.8 | 50.8 | 15.1 |

As can be seen from the result of the examples and test examples, the catalyst according to the present disclosure is capable of producing C₂ hydrocarbons at higher yield than the existing catalysts comprising magnesium oxide or titanium oxide alone. In particular, superior catalytic activity and selectivity were obtained at low temperatures (below 800° C.) when the molar ratio of the oxides was 1:1. When the catalyst having high activity at low temperatures is used, energy consumption in methane reforming can be reduced.

While the present disclosure has been described with respect to the specific embodiments, it will be apparent to those skilled in the art that various changes and modifications may be made without departing from the spirit and scope of the disclosure as defined in the following claims.

1. A catalyst for oxidative coupling of methane comprising: a magnesium titanium oxide support comprising a mixed oxide of magnesium and titanium; and sodium tungstate and manganese oxide supported on the support.

2. The catalyst for oxidative coupling of methane according to claim 1, wherein the magnesium titanium oxide support comprises magnesium and titanium at a molar ratio of from 1:9 to 9:1.

3. The catalyst for oxidative coupling of methane according to claim 1, wherein the magnesium titanium oxide support comprises magnesium and titanium at a molar ratio of 5:5.

4. The catalyst for oxidative coupling of methane according to claim 1, wherein the sodium tungstate is included in an amount of 1-10 wt % and the manganese oxide is included in an amount of 1-5 wt % based on the total weight of the catalyst.

5. The catalyst for oxidative coupling of methane according to claim 1, wherein the sodium tungstate is included in an amount of 3-5 wt % and the manganese oxide is included in an amount of 1-3 wt % based on the total weight of the catalyst.

6. A method for preparing a catalyst for oxidative coupling of methane, comprising:
preparing a solution in which magnesium alkoxide and titanium alkoxide are dissolved in an organic solvent;
inducing hydrolysis and condensation by adding deionized water while stirring the solution, adding a sodium tungstate precursor and a manganese oxide precursor and further stirring until the solution transforms into a gel; and
drying and baking the gel.

7. The method for preparing a catalyst for oxidative coupling of methane according to claim 6, wherein the hydrolysis and the condensation are performed at pH 2-5.

8. The method for preparing a catalyst for oxidative coupling of methane according to claim 6, wherein the hydrolysis, the condensation and the gelation are performed at normal temperature and normal pressure for 6-24 hours.

9. The method for preparing a catalyst for oxidative coupling of methane according to claim 6, wherein the drying is performed at a pressure of 1 torr or lower and the baking is performed at 500-900° C.

10. A method for oxidative coupling of methane, comprising:
packing the catalyst for oxidative coupling of methane according claim 1 in a reactor; and
performing oxidative coupling of methane by introducing a gas mixture comprising methane, oxygen and an inert gas into the reactor.

11. The method for oxidative coupling of methane according to claim 10, wherein the temperature inside the reactor is maintained at 600-900° C.

12. The method for oxidative coupling of methane according to claim 10, wherein the temperature inside the reactor is maintained at 700-850° C.

13. The method for oxidative coupling of methane according to claim 10, wherein the volume ratio of oxygen and methane in the gas mixture is from 1:1 to 1:10.

14. The method for oxidative coupling of methane according to claim 10, wherein the volume ratio of oxygen and methane in the gas mixture is from 1:2 to 1:5.

15. The method for oxidative coupling of methane according to claim 10, wherein the inert gas is helium gas and it is included in the gas mixture in an amount of 10-30 vol %.

16. The method for oxidative coupling of methane according to claim 10, wherein the gas hourly space velocity (GHSV) of the gas mixture inside the reactor is 5,000-30,000 h⁻¹.
